(19) United States
(12) Patent Application Publication
(10) Pub. No.: US 2026/0256833 A1
(43) Pub. Date: Sep. 3, 2026

(54) USE OF BORON FOR THE TREATMENT OF LEISHMANIASIS DISEASE

(71) Applicant: YEDITEPE UNIVERSITESI, Istanbul (TR)

(72) Inventors: Fikrettin SAHIN, Istanbul (TR); Zeynep ISLEK KOKLU, Istanbul (TR); Hazal URAZ, Istanbul (TR); Hatice Ilke OZEKEN, Istanbul (TR)

(73) Assignee: YEDITEPE UNIVERSITESI, Istanbul (TR)

(21) Appl. No.: 19/158,049

(22) PCT Filed: Feb. 20, 2024
(86) PCT No.: PCT/TR2024/050134

§ 371 Date: Aug. 20, 2025

(30) Foreign Application Priority Data
Feb. 20, 2023 (TR) 2023/001871

(51) Int. Cl. A61K 33/22; A61K 45/06; A61P 33/02

(52) U.S. Cl. CPC A61K 33/22; A61K 45/06; A61P 33/02

(57) ABSTRACT

A use of boron derivatives as therapeutics in Leishmaniasis disease is provided. The objective relates to the use of sodium perborate tetrahydrate (SPT), which is almost completely effective on Leishmania parasites, but does not show toxicity in healthy cells, in antileishmanial therapy. Considering the methods and techniques which have been developed, the success achieved in the use of SPT and boron derivatives as therapeutics against Leishmaniasis lays the groundwork for treatments of other infectious diseases and enables the design and development of novel preventive and therapeutic methods. There is provided a novel treatment that can be an alternative to conventional drug therapies (such as amphotericin B antimonials) applied in the treatment of Leishmaniasis, which have difficult patient compliance due to side effects.

### CROSS REFERENCE TO THE RELATED APPLICATIONS

This application is the national phase entry of International Application No. PCT/TR2024/050134, filed on Feb. 20, 2024, which is based upon and claims priority to Turkish Patent Application No. 2023/001871, filed on Feb. 20, 2023, the entire contents of which are incorporated herein by reference.

### TECHNICAL FIELD

The present invention relates to the use of boron as therapeutics in Leishmaniasis disease.

### BACKGROUND

Leishmaniasis is a collective name given for a group of vector-borne diseases caused by protozoan parasites, which are transmitted to humans by the bite of infected female sandflies. Accordingly, the parasite, which exists in amastigote form when the sandfly gets infected by it, develops into a promastigote in the digestive tract of the sandfly and infects the animal or human bitten by the sandfly as promastigote. The promastigote infects macrophages in the body, and it is transformed into amastigotes in macrophages, thereby causing disease.

According to World Health Organization data, Leishmaniasis is widely common in more than 98 countries worldwide, including Turkey and countries geography (in particular, countries in Southern Europe, the Middle East and North Africa) and it has been reported that more than 700 million people are at risk [1, 2, 3]. Clinical manifestations of Leishmaniasis, which is caused by approximately 20 species of Leishmania, are divided into three clinical forms: cutaneous, mucocutaneous, or visceral leishmaniasis (VL) known as ‘Kala Azar’, depending on the site of infection. Visceral Leishmaniasis (VL), colloquially known as Kala Azar Disease, which is a form of leishmaniasis, can be fatal within two years if not untreated. Leishmaia infantum is the main parasite species responsible for this disease in the geographical region of our country and can cause VL.

As is the case with other parasitic diseases, chemotherapy is the most effective method in the treatment of leishmaniasis. However, the high toxicity values of antiparasitic compounds and the drug resistance acquired by parasites over time limit the applicability of chemotherapy. To improve conventional treatment for such infections, there is an urgent need for more effective and selective drugs or drug formulations with low toxicity. The inadequacy of treatment methods has led scientists to try using novel chemotherapeutics in the field of leishmaniasis.

Boron (B) is a trace element with atomic number 5. It can be found in soil, rocks, water, and air. It is widely used in industrial areas. Although it is known that boron has antibacterial effects against several bacteria and fungi, it is not yet known what kind of effect it has on some microorganisms [4]. Recent reports have shown that trace element deficiencies may affect nematode survival and reproduction. It has been reported that infection-induced mineral redistribution may modify host defense, benefit the infectious agents, and promote their survival [5]. It has been observed that a class of boron-containing compounds, the benzoxaboroles, exhibit interesting activities against protozoan pathogens such as Trypanosoma brucei, Plasmodium falciparum, and Trypanosoma cruzi. In addition to the discovery of boronic acids that might be useful as drugs, it has been shown that the heteroaromatic ring system in which the boron atom is incorporated (benzoxaboroles), might be a drug candidate showing anti-inflammatory, antifungal, and antibacterial properties. The fact that boron is incorporated in drug candidate molecules due to its unique properties, which facilitate reversible interactions with biochemical targets, suggests that this element can be used as an antiparasitic molecule [6].

It has been found that boron compounds have inhibitory effects on several cellular components (such as proteasomes, proteases, and peptidases). It has been found that Bortezomib, one of the boron derivatives, which has proteasome inhibitory properties, suppresses both viability and cellular migration in cancer cells [7]. It has been detected that Sodium Perborate Tetrahydrate (SPT) increased the percentages of early and late apoptotic markers more than other types of boron. Therefore, it is believed that SPT may be the most effective type of boron in killing some types of cancer. It has been observed that SPT and some other types of boron (such as boric acid and sodium pentaborate pentahydrate) inhibit cell proliferation in cancerous cells. In addition to its anti-cancer, anti-fungal and anti-bacterial effects, daily boron use has no serious side effects [7].

Leishmaniasis is a parasitic disease widespread in more than 60 countries worldwide, including Turkey and the continental geography (Southern Europe, the Middle East, and North Africa). The combat against leishmaniasis is a matter of national importance today.

As is the case with other parasitic diseases, chemotherapy is the most effective method in the treatment of leishmaniasis. However, the high toxicity values of antiparasitic compounds and the drug resistance acquired by parasites over time limit the applicability of chemotherapy. To improve conventional treatment for such infections, there is an urgent need for more effective and selective drugs or drug formulations with low toxicity.

Treatment of kala azar (VL) is largely based on pentavalent antimonials [8]. The increasing resistance acquired by the parasites against antimonials is the biggest problem limiting the success of this chemotherapy. In fact, in the North Bihar region of India, the success of antimony therapy remains below 50% [9, 10].

Pentavalent antimonials are available as sodium stibogluconate (100 mg/ml) and meglumine antimony (85 mg/ml) and can be administered via i.v. (intravenous) or i.m. (intramuscular). In both cases, equal potency is observed. Usually, 28-day therapies are applied as 20 mg/kg/day. Duration and dose may vary based on clinical syndrome and parasite type. However, it is recommended that the total daily dose should not exceed 850 mg. Amphotericin B (AmB) is preferred in cases where antimonial resistance is observed. This polyene antibiotic has a cure rate close to 100%; however, it is toxic, shows strong side effects and requires long-term hospitalization of the patient [8].

In recent years, therapy with liposomal formulation has become more preferred, especially in Southern Europe [11]. Miltefosine, unlike other therapies against leishmaniasis, is the first therapy method that provides oral treatment and a 3-4-week treatment shows a cure at a level of 95-100% [12, 13]. These data (94%) show a similarity to the results of 6-month Amphotericin B therapy (97%). Its biosafety provides an advantage in one use. Despite its potential for treating large numbers of patients, the primary concern is about the compliance of the drug and possible resistance problems [14]. In summary, pentavalent antimonials (pentostam and glucantime), miltefosine, paromomycin, amphotericin formulated with deoxycholic acid (Fungizone), and amphotericin formulated with liposomes (AmBisome) are currently used in the treatment of leishmaniasis [15]. Except for miltefosine, all other administrations are given via a vein, i.e., intravenous. Furthermore, other treatment limiting challenges are high cost and toxic side effects.

The use of sodium perborate tetrahydrate (SPT), which is a boron derivative, offers several advantages that will reduce these challenges experienced in the treatment of Leishmaniasis. The fact that SPT is taken in daily doses and has no toxic effects on healthy cells reduces unwanted side effects and has the potential to be an effective drug for Leishmaniasis.

It has been observed that the effective dose of SPT on Leishmania parasites does not show toxicity on healthy macrophage cells and does not cause cell death. The use of SPT for the treatment of leishmaniasis features a novel idea.

WO2021124301 A1, an application known in the state of the art, discloses formulations developed for use in the treatment of ocular disorders such as uveitis.

U.S. Pat. No. 7,078,399 B2, an application known in the state of the art, discloses sulfhydryl rifamycin compositions, production methods of the said compositions and methods of treating diseases by using these compositions.

US2010010082 A1, an application known in the state of the art, discloses ophthalmic solutions and methods of using the said solutions to treat ocular disorders.

### SUMMARY

The objective of the present invention relates to the use of sodium perborate tetrahydrate (SPT), a boron derivative, which is almost completely effective on Leishmania parasites, but does not show toxicity in healthy cells, in antileishmanial therapy.

Another objective of the present invention is to provide a novel treatment that can be an alternative to conventional drug therapies (such as amphotericin B, pentavalent antimonials) applied in the treatment of Leishmaniasis, which have difficult patient compliance due to drug resistance and side effects.

### BRIEF DESCRIPTION OF THE DRAWINGS

“Use of Boron for the Treatment of Leishmaniasis Disease”, which is realized to fulfill the objective of the present invention, is illustrated in the accompanying figures, in which:

FIG. 1—is an illustration of the light microscope photographs showing the effect of SPT on the viability of Leishmania infantum parasites in 24, 48 and 72 hours of treatment at different doses (0 (negative control—not incubated with SPT), 25, 50, 75, 100, 125, 150, and 200 μM)).

FIGS. 2A-2B—are the phase-contrast microscopy images of negative control (L. infantum promastigotes not incubated with SPT) and 75, 100 and 150 μM SPT treatment groups after (FIG. 2A) 24 hours and (FIG. 2B) 48 hours. The scale length corresponds to 50 μm.

FIGS. 3A-3C—are the SEM images of L. infantum after 24 hours of incubation with 100 and 200 μM SPT treatment groups at a magnification of (FIG. 3A) 2.5 KX, (FIG. 3B) 5.00 KX, and (FIG. 3C) 7.5 KX. The scale length corresponds to 50 μm.

FIG. 4—is a graphical illustration of the effect of SPT on the mitochondrial membrane potential of L. infantum promastigotes in 24 and 48 hours of treatment at different doses (50-400 μM).

FIG. 5—is a graphical illustration of the effect of SPT on the RAW264.7 health macrophages in 24, 48 and 72 hours of treatment at different doses.

FIG. 6—is a graphical illustration of the infection rate of macrophages infected with L. infantum parasites in 24, 48 and 72 hours of treatment at different doses of SPT.

### DETAILED DESCRIPTION OF THE EMBODIMENTS

The subject matter of the invention relates to boron derivatives (preferably sodium perborate tetrahydrate (SPT)) for the treatment of Leishmaniasis disease, which occurs as a result of infection with Leishmania parasites. Within the scope of the invention, boron derivatives used for providing therapeutic effect to infective cells are administered to macrophage cells which are infected with at least one parasite selected from a group consisting of Leishmania spp. (L. Arabica, L. archibaldi, L. aristedesi, L. braziliensis, L. chagasi, L. colombiensis, L. deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. mexicana, L. naiffi, L. panamensis, L. peruviana, L. pifanoi, L. shawi, L. tarentolae, L. tropica, L. turanica, L. venezuelensis).

Within the scope of the invention, in the use of boron derivatives for the treatment of Leishmaniasis disease, which occurs as a result of infection with Leishmania parasites, a pharmaceutical composition including the said boron derivative is used. This pharmaceutical composition includes extracellular vesicles obtained from macrophages infected with Leishmania parasites and at least one nano-carrier system (selected from a group including emulsion systems, biological and chemical nanoparticles (polymeric nanoparticles, solid lipid nanoparticles), inorganic nanoparticles (metallic nanoparticles), lipid vesicular systems (liposomes, niosomes and ethosomes), dendrimers, polymer-drug conjugates, micelles, and carbon nanotubes). The said pharmaceutical composition includes at least one active compound selected from a group including active compounds showing antiparasitic and/or antineoplastic effect, and binary and ternary combinations thereof, as an active substance. It includes at least one agent selected from a group including nitazoxanide, melarsoprol, eflornithine, metronidazol, tinidazole, miltefosine, mebendazole, pyrantel pamoate, thiabendazole, diethylcarbamazine, ivermectin, niclosamide, praziquantel, albendazole, rifampin, amphotericin B, fumagillin, furazolidone, nifursemizone, nitazoxanide, ornidazole, paromomycin sulfate, pentamidine, pirimethamine, tinidazole, albendazole, mebendazole, thiabendazole, fenbendazole, triclabendazole, flubendazole, abamectin, diethylcarbamazine, ivermectin, suramin, pyrantel pamoate, levamisole, niclosamide, nitazoxanide, oxyclozanide, monepantel, derquantel, amphotericin B, urea stibamine, sodium stibogluconate, meglumine antimoniate, paromomycin, miltefosine, fluconazole, pentamidine, bisnaphthalimidopropyl (BNIP) derivatives, and binary or ternary combinations and encapsulations thereof, as active compounds showing antiparasitic effect. Also, it includes at least one agent selected from a group including cyclophosphamide, ifosfamide, temozolomide, capecitabine, 5-fluorouracil, methotrexate, gemcitabine, pemetrexed, mitomycin, bleomycin, epirubicin, doxorubicin, etoposide, paclitaxel, irinotecan, docetaxel, vincristine, carboplatin, cisplatin, oxaliplatin, bevacizumab, cetuximab, gefitinib, imatinib, trastuzumab, denosumab, rituximab, sunitinib, zoledronate, abiraterone, anastrozole, bicalutamide, exemestane, goserelin, medroxyprogesterone, octreotide, tamoxifen, bendamustine, carmustine, chlorambucil, lomustine, melphalan, procarbazine, streptozocin, fludarabine, raltitrexed, actinomycin D, dactinomycin, doxorubicin, mitoxantrone, eribulin, topotecan, vinblastine, vinorelbine, afatinib, aflibercept, crizotinib, dabrafenib, interferon, ipilimumab, lapatinib, nivolumab, panitumumab, pembrolizumab, pertuzumab, sorafenib, trastuzumab emtansine, temsorilimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, vincristine, and binary or ternary combinations and encapsulations thereof, as active compounds showing antineoplastic effect in combination with extracellular vesicles and/or nano-carrier systems.

Pharmaceutical composition of the present invention can be administered by at least one administration method selected from a group consisting of parenteral, intravenous, intradermal, subcutaneous, intraperitoneal, topical, intrathecal, intranasal, intracerebroventricular, ocular, vaginal, urethral, transdermal, sublingual, subarachnoid, rectal, periodontal, perineural, peridural, periarticular, oral, intratympanic, intratumor, intrapulmonary, intrasynovial, intramuscular, intraovarian, intrameningeal, intracorporus cavernosum, intracoronary, intracerebral, epidural, cutaneous, buccal, dental administration methods.

A pharmaceutical composition for use in the treatment of Leishmaniasis according to the claims includes an adjuvant which is at least one selected from the group consisting of monophosphoryl lipid A (MPL), cholesterol, CG oligonucleotide-containing aluminum hydroxide, aluminum phosphate, tocopherol, emulsion systems, or binary or more combinations thereof. The said components used as adjuvant are used alone or in combination with the other agents listed above in the treatment of Leishmaniasis.

Inactivated vaccines produced from dead microorganisms do not show antigenic proliferation; for the dead vaccines showing a lower immunogenic response to be able to create immunization, they should be repeated with multiple doses at regular intervals and administered in conjunction with the adjuvant. Adjuvants are substances that are themselves non-immunogenic, and do not form antibodies, but increase and strengthen the immunogenicity of the antigen to which they are administered. In relatively less purified vaccines in which entire dead microorganism is used, some components of the microorganism (such as endotoxins) may act as adjuvants (intrinsic adjuvants). These vaccines, which also contain “intrinsic adjuvants” in addition to the normal adjuvant added to the vaccine, have the effect of increasing the immunity of both their own antigens and other antigens that they are administered together (such as diphtheria and tetanus vaccines containing whole-cell pertussis vaccine) [16].

Monophosphoryl lipid A (MPL®) is the first vaccine adjuvant to achieve clinical and market success since the introduction of aluminum salts in the early 20th century. First, a component of adjuvant system 4 [AS04 (1)], aluminum hydroxide semi-crystalline gels that are hydrostatically adsorbed with MPL, was approved for use in the HBV vaccine Fendrix (2) in patients with renal failure and then for more extensive use (e.g., HPV vaccine Cervarix). Completely aluminum-free formulations, such as adjuvant system 1 containing MPL and QS-21 in liposomal complexes, have achieved similar success as the adjuvant component of Shingrix, which is a varicella zoster vaccine. Since MPL® is a highly purified derivative of the lipopolysaccharide (LPS) component of the cell wall of Salmonella enterica, its success as an adjuvant is recognized mainly in terms of its activity as a TLR4 agonist which directly activates dendritic cells [17].

In biocompatible and biodegradable, spherical (round) polymeric systems with nanometer-micrometer sizes, poly (DL-lactide-co-glycolide) microspheres can adsorb and carry many different types of long antigens. Polylactide co-glycolide (PLG) microparticle is one of the most commonly used polymeric microspheres. By means of the cationic or anionic PLG preparations, various types of antigens (plasmid DNA, recombinant protein, immunostimulant oligonucleotides) are adsorbed and presented to antigen-presenting cells. In this way, a much stronger immune response is obtained compared to aluminum. It has been found that many adjuvants and immunostimulants, such as non-methylated bacterial/viral CpG DNA and oligonucleotides (TLR9), LPSs and derivatives thereof (TLR4), lipopeptides and tripalithoyl-S-glyceryl cysteine (TLR2), imidazoquinolone (TLR7/TLR8) are Toll-like receptor (TLR) agonists [16].

The subject matter of the invention relates to the use of sodium perborate tetrahydrate (SPT), a boron derivative, in the treatment of Leishmaniasis disease, which occurs as a result of infection with Leishmania parasites. The objective of the invention is to provide a novel treatment method as therapeutics to the infective cells, by administering sodium pentaborate tetrahydrate to macrophage cells infected with Leishmania. The fact that macrophage cells infected with Leishmania parasites causing this disease in the geographical region of our country can be treated with SPT may provide an alternative therapy method to currently available chemotherapy methods which are insufficient in treatment. Within the scope of this antileishmanial therapy, it has been observed that SPT is highly lethal to Leishmania parasites, but it shows no side effects on healthy macrophage cells, following the administration of Sodium perborate tetrahydrate to parasite-infected macrophages.

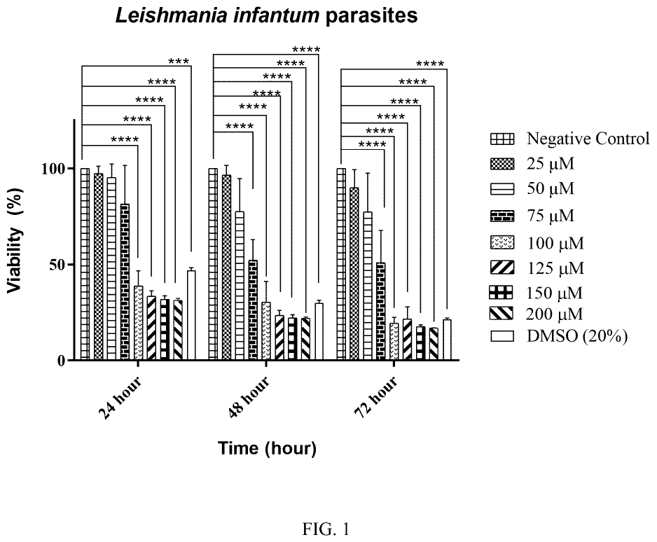

L. infantum promastigotes were treated with SPT at concentrations ranging from 25 μM to 200 μM for 24, 48 and 72 hours, and the effect of SPT on cell viability of parasites was determined. As shown in the FIG. 1, SPT concentrations of 75, 100, 125, 150 and 200 μM inhibited the proliferation of parasites in a time-dependent manner at 24, 48 and 72 hours. When L. infantum was treated with SPT at concentrations of ≤50 uM, cell viability remained above 80% after 24, 48 and 72 hours of incubation. When the concentration was increased to 75 μM, cell viability was reduced to below 80% (P≤0.0001) and 55% (P≤0.0001) at 24 and 48 hours, respectively; when the concentration was increased above 100 μM, the parasite viability was detected to be below approximately 33% and 25%. Furthermore, after 72 hours of incubation, SPT concentration of 75 μM significantly reduced the viability of L. infantum promastigotes to 50% (P≤0.0001) compared to the negative control; the increase in SPT concentration to 100, 125, 150, and 200 μM led to a significant reduction in parasite viability by approximately 80% (P≤0.0001).

IC50 values for SPT were calculated as 71.99±7.51 μM in 72 hours. The results have demonstrated that SPT is more effective against L. infantum promastigotes at 48 and 72 hours of incubation than 24 hours of incubation with a lower IC50 value.

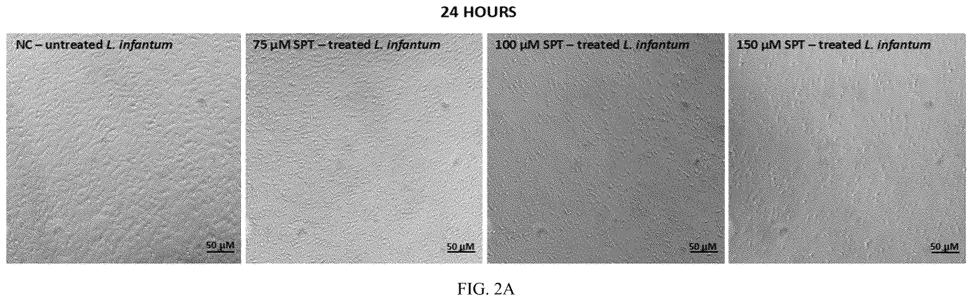

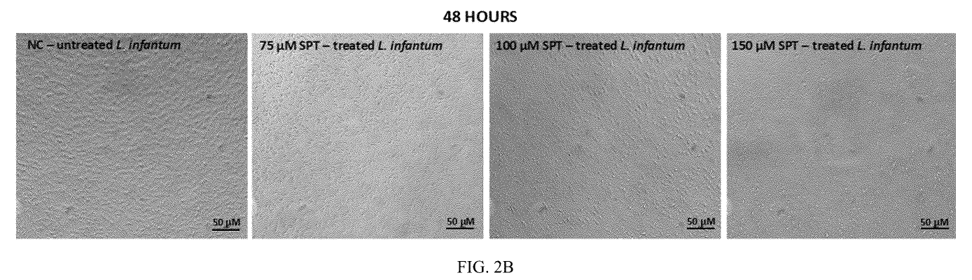

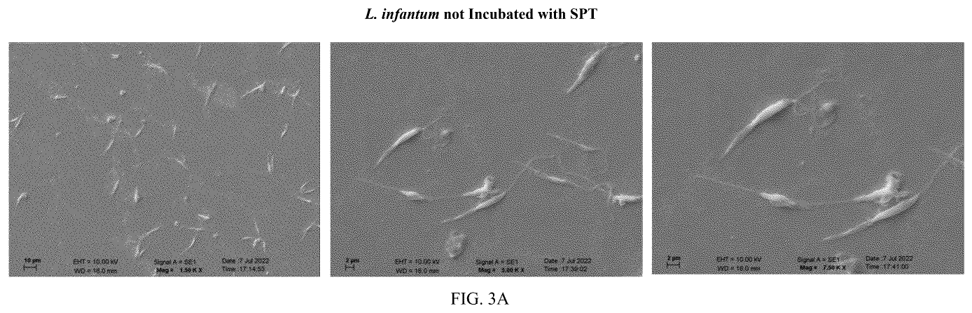

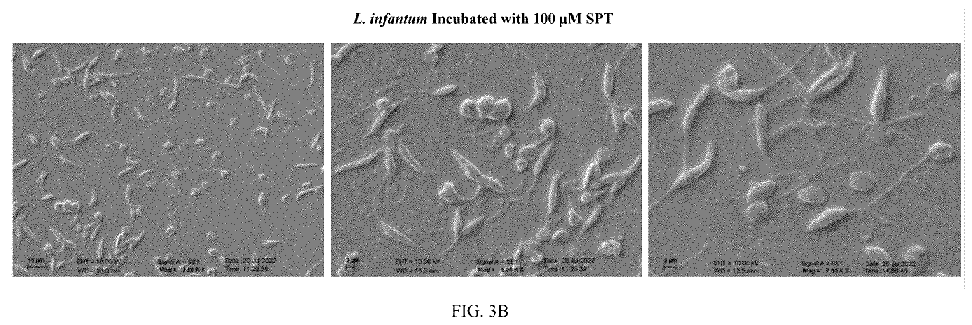

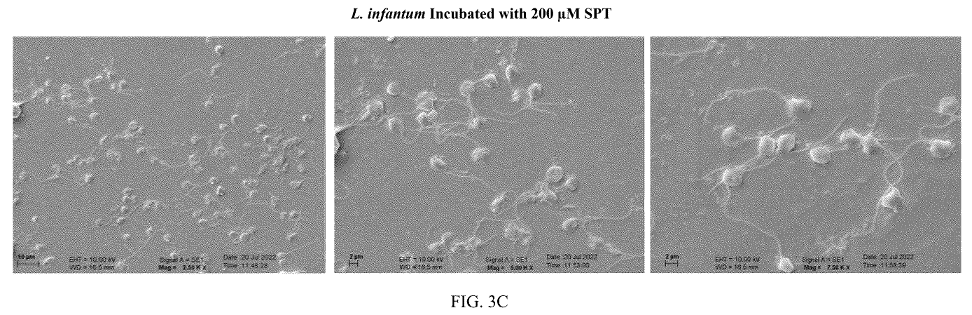

In consistency with the parasite viability data, as shown in the phase-contrast microscopy images in FIGS. 2A-2B and scanning electron microscopy (SEM) images in FIGS. 3A-3C, after 24 and 48 hours of incubation with SPT, a dose-dependent decrease in cell number was detected, and the flagellated structure of the parasites changed from an elongated to a rounded shape (FIGS. 2A-2B and FIGS. 3A-3C). This was attributed to the loss of membrane and flagellum integrity of L. infantum promastigotes by SPT which causes apoptosis. Also, morphological changes such as spherical shape and shrinkage showed strong signs of apoptosis. When the SPT concentration was increased to ≥75 μM, an increase in the number of parasites which had lost their integrity was observed (FIGS. 2A-2B and FIGS. 3A-3C).

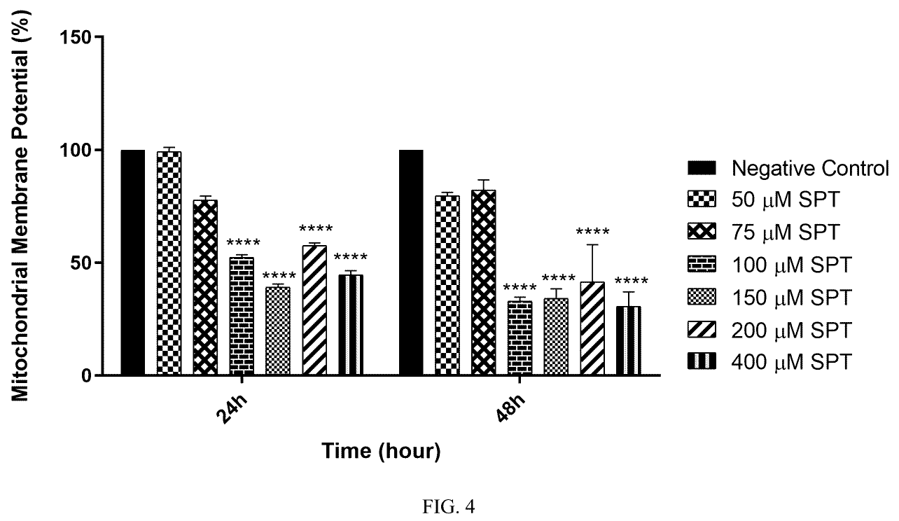

After incubation of L. infantum parasites with different doses of SPT (50-400 μM) for 24 and 48 hours, parasite mitochondrial membrane potential was determined by microplate reader and FACS analysis (FIG. 4). According to the results obtained, a time-dependent decrease in the mitochondrial membrane potential of promastigotes was determined. When L. infantum was treated with SPT at concentrations of ≤75 uM, the percentage of mitochondrial membrane potential remained above 80% after 24 and 48 hours of incubation; when treated with SPT at concentrations of ≥100 μM, L. infantum promastigotes caused a significant decrease in mitochondrial membrane potential (below 53% after 24 hours and below 35% after 48 hours) compared to the negative control group (p≤0.0001). As shown in FIG. 4, 24 hours incubation of L. infantum promastigotes with 100, 150, 200 and 400 μM SPT significantly reduced mitochondrial membrane potential (52.2%, 39.1%, 57.5% and 44.6%, respectively) compared to parasites not incubated (96.16%). Furthermore, the mitochondrial membrane potential percentages, which were found to be 32.8%, 34.1%, 41.5%, and 30.1% after 48 hours of 100, 150, 200, and 400 μM SPT incubation, are given in FIG. 4.

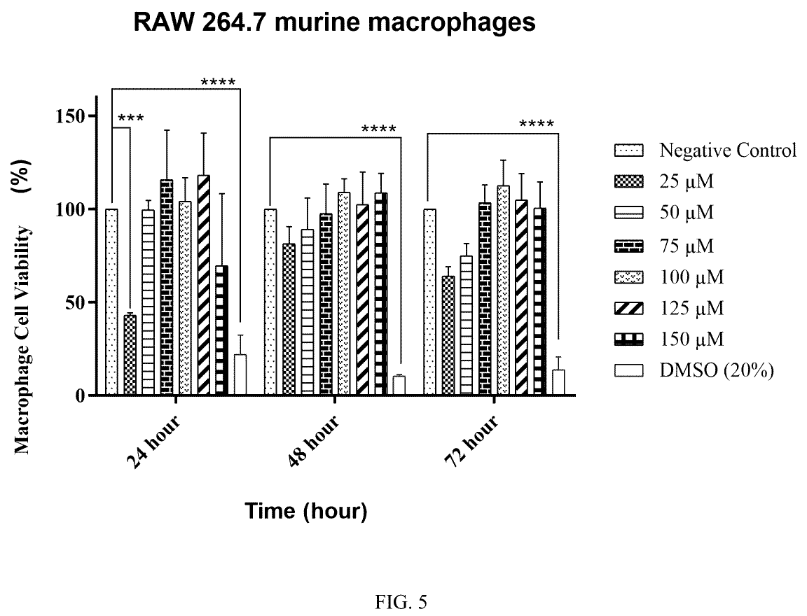

Following the in vitro evaluation of the antileishmanial effect of SPT on L. infantum promastigotes, the effect of SPT on cell viability on healthy RAW264.7 macrophages is shown in FIG. 5. Accordingly, when uninfected macrophages were incubated with SPT at concentrations of ≥75 μM, the cell viability of their macrophages was determined to be above 90% for 24, 48 and 72 hours. When the concentration was reduced to 50 μM, cell viability remained above 90% for 24 hours and 48 hours; it became exactly 75% after 72 hours compared to the negative control group. The increase of the SPT concentration above 100 μM did not show any toxicity on uninfected macrophages; the obtained cell viability data demonstrated that SPT concentrations of 100, 75, 100, 125 and 150 μM did not show any toxicity on healthy macrophages, and they have safety potential for antileishmanial therapy.

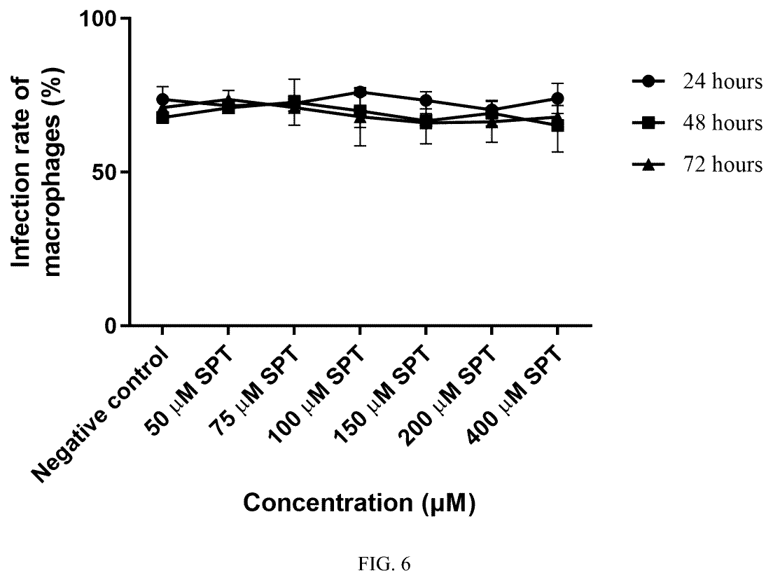

For the antileishmanial activity of SPT on macrophages infected with Leishmania, its concentrations of 50, 75, 100, 150, 200 and 400 μM were tested, and after 24, 48, and 72 hours, infection rates were determined (FIG. 6). Flow cytometry data demonstrated that when macrophages were incubated with L. infantum promastigotes, 73.67% of macrophages were infected (FIG. 6). As a result of incubation of macrophages infected with Leishmania with ≥50 μM SPT (i.e., 50, 75, 100, 150, 200 and 400 μM) for 24, 48 and 72 hours, it was observed that the infection rate did not increase regardless of time and concentration, and it was determined that infection rates were kept within a certain limit.

Considering the methods and techniques which have been developed, it is believed that the success achieved in the use of SPT and boron derivatives as therapeutics against Leishmaniasis observed within the scope of the invention, lays the groundwork for treatments of other infectious diseases and enables the design and development of novel preventive and therapeutic methods. The fact that boron intake at daily doses does not cause any side effects in the body and the parasites will not develop resistance to the drug will enable boron to serve as a potentially effective drug against leishmaniasis disease. It will provide basis for novel molecules that can be an alternative to conventional drug therapies (such as amphotericin B antimonials) applied in the treatment of Leishmaniasis, which have difficult patient compliance due to side effects.

### DESCRIPTION OF EXPERIMENTAL STUDY

In the experimental studies conducted within the scope of the invention, SPT alone was first tested on Leishmania parasites and then on macrophages infected with Leishmania. Since it is desired to test an antileishmanial effect and Leishmania is an obligate intracellular parasite, testing must be performed in macrophages. In this regard, experimental studies were conducted in in vitro cell culture in order to determine its efficacy on Leishmania parasites within 24, 48 and 72 hours. It has been detected to be highly effective on Leishmania parasites, and it has been observed that it does not show any side effects on healthy cells.

### 1. Culturing the Parasites

Leishmania infantum (MHOM/MA/67/ITMA-P263) promastigotes are incubated in RPMI medium (heat inactivated 10% fetal bovine serum, 2 mM L-glutamine, 20 mM HEPES, 100 U/ml penicillin, 100 μg/ml streptomycin) at 27° C. Parasites reaching the logarithmic phase (10⁶/ml) are made infective.

### 2. Antileishmanial Use of SPT on Leishmania Parasites: Resazurin Assay

Leishmania parasites are incubated in culture medium and the effect of SPT on the proliferation of their promastigotes is analyzed. In summary, Leishmania infantum (MHOM/MA/67/ITMA-P263) promastigotes are incubated in RPMI medium (heat inactivated 10% fetal bovine serum, 2 mM L-glutamine, 20 mM Hepes, 100 U/ml penicillin, 100 μg/ml streptomycin) at 27° C. Parasites reaching the logarithmic phase (10⁶/ml), after 2 days of incubation, are incubated at 27° C. for 3 days with SPT at different concentration ranges. The viability of the parasites is determined by applying Alamar Blue assay. By obtaining IC₅₀ values from the samples whose fluorescence intensities are read according to the Alamar Blue assay protocol, the activities of SPT are determined.

In parallel to this, Leishmania parasites incubated with SPT at different concentrations were morphologically examined under scanning electron microscopy.

3. Measuring the Mitochondrial Membrane Potential of Leishmania Parasites Incubated with SPT at Different Concentrations

Mitochondrial membrane potential (ΔΨPm) is measured by using Rhodamine 123 mitochondrial specific fluorescent dye (Sigma-Aldrich). In summary, cells were seeded in a 48-well plate at 300.000 parasites/well. The following day, parasites are treated with SPT at various concentrations (50-400 uM) for 24 and 48 hours. After incubation, cells are resuspended in serum-free RPMI containing 10 μM Rhodamine 123 and incubated in the dark at 37-C for 30 minutes. They are then centrifuged and washed twice with 1×DPBS. The cell suspension is transferred to a 96-well plate and fluorescence intensities are measured at 490 nm excitation and 515 nm emission by using a spectrophoto-fluorimeter (Varioskan LUX multimode reader, Thermo Scientific). Absorbance is evaluated as a fold change with respect to the negative control group.

### 4. Culturing the Macrophage

Macrophage RAW264.7 cell line (ATCC) is grown as monolayer in RPMI 1640 nutrient medium (2 mM L-glutamine, 100 U/ml penicillin, 100 g/ml streptomycin) with 10% FBS, heat inactivated in a humidified atmosphere environment with 5% CO₂ at 37° C. Cells are passaged at 3-day intervals.

5. Treating Healthy Macrophages with SPT and Determining Cell Viability

Cells were seeded in 96-well culture dishes (Corning Glasswork, Corning, NY) in Dulbecco's modified Eagle's medium (DMEM) containing 10% fetal bovine serum (Invitrogen) and 1% PSA (Biological Industries, Beit Haemek, Israel) at 20.000 cells/well and then treated with SPT, and cell viability levels were measured on days 1, 2 and 3. Cell viability is measured by using 3-(4,5-di-methyl-thiazol-2-yl)-5-(3-carboxy-methoxy-phenyl)-2-(4-sulfo-phenyl)-2H-tetrazolium (MTS)-method (CellTiter96 AqueousOne Solution; Promega, Southampton, UK). 10 μl MTS solution is added onto the cells in 100 μl growth medium and they are incubated in the dark for 2 hours. After the incubation period, viability analysis is obtained by performing absorbance measurement with an ELISA plate reader (Biotek, Winooski, VT) at 490 nm wavelength.

6. Treating Infected Macrophages with SPT and Determining the Infection Rate
Staining the Parasites and Macrophages with PKH Lipid Membrane Dyes

RAW264.7 macrophage cells and L. infantum promastigotes were stained with PKH26 (Red Fluorescent Cell Linker Kit, Sigma-Aldrich) and PKH67 (Green Fluorescent Cell Linker Kit, Sigma-Aldrich), respectively, according to the procedure described previously in the article by Islek et al. (2021). A total cell concentration of 10⁷ cells/ml was used. Before infection, the staining rate of parasites and cells was determined by flow cytometry.

### Determining the Infection Rate

The effect of SPT on the proliferation of macrophage cells infected with parasites is analyzed. In summary, macrophages are infected with parasites at 37° C. at a ratio of 10:1 (parasite: macrophage). After 3 and a half hours, infected macrophages are washed with medium to remove the remaining parasites. Infected macrophages are left to incubate with SPT at different concentration ranges at 37° C. for 3 days, fixed at the end of 3 days and the infection rate is determined by flow cytometer. The percentage of infection is determined according to the following formula:

$$Percentage of infection = \frac{Macrophage infected with the parasite}{Total macrophage} \times 100$$

### REFERENCES

[1]. Svobodova, M., Alten, B., Zidkova, L., Dvorak, V., Hlavackova, J., Myskova, J., . . . Volf, P. (2009). Cutaneous leishmaniasis caused by Leishmania infantum transmitted by Phlebotomus tobbi. Int J Parasitol, 39(2), 251-256. doi.org/S0020-7519(08)00270-1 [pii]\r10.1016/j.ijpara.2008.06.016
[2]. Ok, Ü. Z., Balcioğlu, İ. C., Taylan Özkan, A., Özensoy, S. & Özbel, Y. (2002). Leishmaniasis in turkey. Acta Tropica, 84, 43-48
[3]. Tanir, G., Taylan Özkan, A., & Daǧlar, E. (2006). Pediatric visceral Leishmaniasis in Turkey. Pediatrics International, 48(1), 66-69. doi.org/10.1111/j.1442-200X.2006.02153.x
[4]. Sayin, Z., Ucan, U. S., & Sakmanoglu, A. (2016). Antibacterial and antibiofilm effects of boron on different bacteria. Biological trace element research, 173(1), 241-246.
[5]. Bourgeois, A. C., Scott, M. E., Sabally, K., & Koski, K. G. (2007). Low dietary boron reduces parasite (nematoda) survival and alters cytokine profiles but the infection modifies liver minerals in mice. The Journal of nutrition, 137(9), 2080-2086.
[6]. Jacobs, R. T., Plattner, J. J., & Keenan, M. (2011). Boron-based drugs as antiprotozoals. Current opinion in infectious diseases, 24(6), 586-592.
[7]. Cebeci, E., Yüksel, B., Şahin, F. (2022). Anti-cancer effect of boron derivatives on small-cell lung cancer. Journal of Trace Elements in Medicine and Biology, 70, 126923.
[8]. Sundar, S. & Chakravarty, J. 2013. Leishmaniasis: An update of current pharmacotherapy. Expert Opinion on Pharmacotherapy, 14, 53-63.
[9]. Murray, H. W., Berman, J. D., Davies, C. R. & Saravia, N. G. 2005. Advances in leishmaniasis. Lancet, 366, 1561-77.
[10]. Sundar, S., Chatterjee, M. (2006). Visceral leishmaniasis—current therapeutic modalities. Indian J Med Res, 123, 345-52.
[11]. Piscopo, T. V, & Mallia Azzopardi, C. (2007). Leishmaniasis. Postgraduate Medical Journal, 83(976), 649-57. doi.org/10.1136/pgmj.2006.047340corr1
[12]. Sundar, S., Makharia, A., More, D. K., Agrawal, G., Voss, A., Fischer, C., Bachmann, P. & Murray, H. W. 2000. Short-course of oral miltefosine for treatment of visceral leishmaniasis. Clinical Infectious Diseases, 31, 1110-1113.
[13]. Jha, T. K., Sundar, S., Thakur, C. P., Bachmann, P., Karbwang, J., Fischer, C., Voss, A. & Berman, J. 1999. Miltefosine, an oral agent, for the treatment of indian visceral leishmaniasis. New England Journal of Medicine, 341, 1795-1800.
[14]. Murray, H. W., Berman, J. D., Davies, C. R. & SARAVIA, N. G. 2005. Advances in leishmaniasis. Lancet, 366, 1561-77.
[15]. Croft, S. L. & Olliaro, P. 2011. Leishmaniasis chemotherapy-challenges and opportunities. Clinical Microbiology and Infection, 17, 1478-1483.
[16]. Yurdakök, K., and İnce, T. (2008). Aşi adjuvanlari. Çocuk Sağlιğι ve Hastalιklarι Dergisi. 51: 225-239
[17]. Wang Y Q, Bazin-Lee H, Evans J T, Casella C R, Mitchell T C. MPL Adjuvant Contains Competitive Antagonists of Human TLR4. Front Immunol. 2020 Oct. 16; 11:577823. doi: 10.3389/fimmu.2020.577823

What is claimed is:

1. A boron derivative used in a treatment of a Leishmaniasis disease, wherein the Leishmaniasis disease occurs as a result of an infection with Leishmania parasites.

2. The boron derivative according to claim 1, wherein the boron derivative is sodium perborate tetrahydrate (SPT).

3. The boron derivative according to claim 1, wherein the boron derivative is configured for providing a therapeutic effect to infective cells by being administered to macrophage cells, wherein the macrophage cells are infected with at least one parasite selected from the group consisting of L. Arabica, L. archibaldi, L. aristedesi, L. braziliensis, L. chagasi, L. colombiensis, L. deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. mexicana, L. naiffi, L. panamensis, L. peruviana, L. pifanoi, L. shawi, L. tarentolae, L. tropica, L. turanica, and L. venezuelensis.

4. A pharmaceutical composition, comprising the boron derivative according to claim 1.

5. The pharmaceutical composition according to claim 4, further comprising:
extracellular vesicles, wherein the extracellular vesicles are obtained from macrophage cells infected with the Leishmania parasites, and
nano-carries systems, wherein the nano-carries systems are selected from the group consisting of the following:
emulsion systems,
biological nanoparticles and chemical nanoparticles, comprising polymeric nanoparticles and solid lipid nanoparticles,
inorganic nanoparticles comprising metallic nanoparticles,
lipid vesicular systems comprising liposomes, niosomes, and ethosomes,
dendrimers,
polymer-drug conjugates micelles, and
carbon nanotubes.

6. The pharmaceutical composition according to claim 4, further comprising at least one active compound selected from the group consisting of an active compound showing an antiparasitic effect and/or an active compound showing an antineoplastic effect, a binary combination thereof, and a ternary combination thereof, as an active substance.

7. The pharmaceutical composition according to claim 6, wherein the active compound showing the antiparasitic effect is at least one selected from the group consisting of nitazoxanide, melarsoprol, eflornithine, metronidazol, tinidazole, miltefosine, mebendazole, pyrantel pamoate, thiabendazole, diethylcarbamazine, ivermectin, niclosamide, praziquantel, albendazole, rifampin, amphotericin B, fumagillin, furazolidone, nifursemizone, nitazoxanide, ornidazole, paromomycin sulfate, pentamidine, pirimethamine, tinidazole, albendazole, mebendazole, thiabendazole, fenbendazole, triclabendazole, flubendazole, abamectin, diethylcarbamazine, ivermectin, suramin, pyrantel pamoate, levamisole, niclosamide, nitazoxanide, oxyclozanide, monepantel, derquantel, amphotericin B, urea stibamine, sodium stibogluconate, meglumine antimoniate, paromomycin, miltefosine, fluconazole, pentamidine, bisnaphthalimidopropyl (BNIP) derivatives, a binary combination thereof or a ternary combination thereof, and encapsulations thereof.

8. The pharmaceutical composition according to claim 6, wherein the active compound showing the antineoplastic effect is in combination with extracellular vesicles and/or nano-carrier systems, and is at least one selected from the group consisting of cyclophosphamide, ifosfamide, temozolomide, capecitabine, 5-fluorouracil, methotrexate, gemcitabine, pemetrexed, mitomycin, bleomycin, epirubicin, doxorubicin, etoposide, paclitaxel, irinotecan, docetaxel, vincristine, carboplatin, cisplatin, oxaliplatin, bevacizumab, cetuximab, gefitinib, imatinib, trastuzumab, denosumab, rituximab, sunitinib, zoledronate, abiraterone, anastrozole, bicalutamide, exemestane, goserelin, medroxyprogesterone, octreotide, tamoxifen, bendamustine, carmustine, chlorambucil, lomustine, melphalan, procarbazine, streptozocin, fludarabine, raltitrexed, actinomycin D, dactinomycin, doxorubicin, mitoxantrone, eribulin, topotecan, vinblastine, vinorelbine, afatinib, aflibercept, crizotinib, dabrafenib, interferon, ipilimumab, lapatinib, nivolumab, panitumumab, pembrolizumab, pertuzumab, sorafenib, trastuzumab emtansine, temsorilimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, vincristine, a binary combination thereof or a ternary combination thereof, and encapsulations thereof.

9. The pharmaceutical composition according to claim 4, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavernosum administration method, an intracoronary administration method, an intracerebral administration method, an epidural administration method, a cutaneous administration method, a buccal administration method, and a dental administration method.

10. The pharmaceutical composition according to claim 4, further comprising an adjuvant, wherein the adjuvant is at least one selected from the group consisting of monophosphoryl lipid A (MPL), cholesterol, CG oligonucleotide-containing aluminum hydroxide, aluminum phosphate, tocopherol, emulsion systems, and a binary thereof or more combinations thereof, wherein the pharmaceutical composition is used in the treatment of the Leishmaniasis disease.

11. The pharmaceutical composition according to claim 4, wherein the boron derivative is SPT.

12. The pharmaceutical composition according to claim 4, wherein the boron derivative is configured for providing a therapeutic effect to infective cells by being administered to macrophage cells, wherein the macrophage cells are infected with at least one parasite selected from the group consisting of L. arabica, L. archibaldi, L. aristedesi, L. braziliensis, L. chagasi, L. colombiensis, L. deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. mexicana, L. naiffi, L. panamensis, L. peruviana, L. pifanoi, L. shawi, L. tarentolae, L. tropica, L. turanica, and L. venezuelensis.

13. The pharmaceutical composition according to claim 5, further comprising at least one active compound selected from the group consisting of an active compound showing an antiparasitic effect and/or an active compound showing an antineoplastic effect, a binary combination thereof, and a ternary combination thereof, as an active substance.

14. The pharmaceutical composition according to claim 5, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavernosum administration method, an intracoronary administration method, an intracerebral administration method, an epidural administration method, a cutaneous administration method, a buccal administration method, and a dental administration method.

15. The pharmaceutical composition according to claim 6, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavernosum administration method, an intracoronary administration method, an intracerebral administration method, an epidural administration method, a cutaneous administration method, a buccal administration method, and a dental administration method.

16. The pharmaceutical composition according to claim 7, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavernosum administration method, an intracoronary administration method, an intracerebral administration method, an epidural administration method, a cutaneous administration method, a buccal administration method, and a dental administration method.

17. The pharmaceutical composition according to claim 8, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavernosum administration method, an intracoronary administration method, an intracerebral administration method, an epidural administration method, a cutaneous administration method, a buccal administration method, and a dental administration method.

18. The pharmaceutical composition according to claim 5, further comprising an adjuvant, wherein the adjuvant is at least one selected from the group consisting of MPL, cholesterol, CG oligonucleotide-containing aluminum hydroxide, aluminum phosphate, tocopherol, emulsion systems, and a binary thereof or more combinations thereof, wherein the pharmaceutical composition is used in the treatment of the Leishmaniasis disease.

19. The pharmaceutical composition according to claim 6, further comprising an adjuvant, wherein the adjuvant is at least one selected from the group consisting of MPL, cholesterol, CG oligonucleotide-containing aluminum hydroxide, aluminum phosphate, tocopherol, emulsion systems, and a binary thereof or more combinations thereof, wherein the pharmaceutical composition is used in the treatment of the Leishmaniasis disease.

20. The pharmaceutical composition according to claim 7, further comprising an adjuvant, wherein the adjuvant is at least one selected from the group consisting of MPL, cholesterol, CG oligonucleotide-containing aluminum hydroxide, aluminum phosphate, tocopherol, emulsion systems, and a binary thereof or more combinations thereof, wherein the pharmaceutical composition is used in the treatment of the Leishmaniasis disease.
